(19) United States
(12) Patent Application Publication
(10) Pub. No.: US 2023/0293459 A1
(43) Pub. Date: Sep. 21, 2023

(54) OLEYLCYSTEINEAMIDE OR DERIVATIVES THEREOF AND THEIR USE IN THERAPY

(71) Applicants: YISSUM RESEARCH DEVELOPMENT COMPANY OF THE HEBREW UNIVERSITY OF JERUSALEM LTD., Jerusalem (IL); HADASIT MEDICAL RESEARCH SERVICES & DEVELOPMENT LIMITED, Jerusalem (IL)

(72) Inventors: Simon BENITA, Tel Aviv (IL); Taher NASSAR, Kfar Tur'an (IL); Riki PERLMAN, Beit Zayit (IL); Dina BEN YEHUDA, Mevaseret Zion (IL); Ihab ABD-ELRAHMAN, Abu Gosh (IL); Noha KHAIRI, Beit Hanina (IL)

(21) Appl. No.: 18/040,450

(22) PCT Filed: Aug. 5, 2021
(86) PCT No.: PCT/IL2021/050955

§ 371 Date: Feb. 3, 2023

Related U.S. Application Data
(60) Provisional application No. 63/062,292, filed on Aug. 6, 2020.
(60) Provisional application No. 63/165,390, filed on Mar. 24, 2021.

(51) Int. Cl. A61K 31/16; A61K 47/59; A61K 9/00; A61P 27/02; A61K 8/46; A61Q 19/02

(52) U.S. Cl. CPC A61K 31/16; A61K 47/593; A61K 9/0014; A61P 27/02; A61K 8/46; A61Q 19/02

(57) ABSTRACT

The subject-matter of the present application concerns oleylcysteineamide (OCA) and OCA derivatives as active agents and their use in the treatment of inflammatory disorders as well as in treating skin whitening.

### TECHNOLOGICAL FIELD

The invention generally concerns novel adjuvants and anti-inflammatory agents and uses thereof.

### BACKGROUND

Immunotherapy has emerged as an effective strategy for the prevention and treatment of a variety of diseases, including cancer, infectious diseases, inflammatory diseases, and autoimmune diseases. In the treatment of cancer and infectious diseases, immunostimulatory therapy should be used for the activation of immune response to detect and eliminate non-self-antigens, and to establish memory effects for these diseases. On the contrary, for overactive immune response in diseases like atherosclerosis, rheumatoid arthritis (RA), diabetes, obesity, and transplantation, immunosuppressive therapy is needed to downregulate immune reaction and generate certain immune tolerance. The mammalian immune environment can be regulated by a variety of immune cells, cytokines, and enzymes, which shave been shown to control and prevent immune-related disorders or illnesses. Many immunotherapeutic methods have achieved impressive outcomes in treating various diseases, but performances of immunoregulatory agents can be negatively affected by poor solubility, high immune-mediated toxicity, and loss of bioactivity after long circulation.

Immunomodulatory agents and drugs incorporated into nano-delivery system, have been shown to improve the therapeutic effects and simultaneously overcome many obstacles facing other treatment methods, such as inadequate immune stimulation, off-target side effects, and bioactivity loss of immune agents during circulation. In recent years, researchers have continuously developed nanomaterials with new structures, properties, and functions. In cancer immunotherapy, nano-systems have been shown to play an essential role in immune cell activation and tumor microenvironment modulation. In infectious diseases, many encouraging outcomes from using nanomaterial vaccines against viral and bacterial infections have been reported. In addition, nanoparticles have been shown to potentiate the effects of immunosuppressive immune cells for the treatment of inflammatory and autoimmune diseases.

Oleylcysteineamide (OCA) has been used as a linker moiety for associating various active agents to a surface region of a particle carrier. The use of OCA as a linker moiety has increased delivery efficacy of the active agent. No measurable effect was demonstrated for neither for OCA itself nor for an OCA-associated particle.

### BACKGROUND ART

[1] WO12101638
[2] WO12101639

### GENERAL DESCRIPTION

Theoretically, our immune system is able to protect us from a variety of illnesses through a natural ‘immune surveillance’, by which viruses, bacteria, and cancer cells can be rapidly identified as alien antigens and eliminated by immune cells. In reality, however, successful pathogens have developed a range of effective mechanisms to evade immune clearance by inhibiting phagocytosis, blocking antigen presentation, or directly killing immune cells. Worse still, cancer cells can alter the tumor microenvironment into a highly immunosuppressive state by recruiting immunosuppressive immune cells and by expressing a series of inhibitory cytokines, enzymes, and checkpoint molecules, thus facilitating tumor immune evasion. These barriers hinder the efficiency and intensity of the natural immune responses. On the contrary, aberrant activation of immune cells can arouse uncontrolled inflammation and cause inflammatory diseases, autoimmune diseases, or allergic diseases. Abnormal inflammation can also lead to transplant rejection and hinder tissue and organ regeneration.

Therefore, on one hand, the use of external immunomodulators is necessary to assist and overcome the pitfalls of the natural immune system. On the other hand, their use requires careful control and adaptability so as to maintain the homeostasis and function of the immune system.

A large number of synthetic derivatives, organic and inorganic compounds and naturally occurring substances are able to suppress, modulate or enhance the immune response. Immunomodulators, some of which are chemically well-defined and others that are complex preparations, exhibit a great variety of structures and immuno-pharmacological properties. Essentially, these molecules act on a host own immune system to fight immunocompromised conditions, such as cancer and others as above.

The present invention is based on the surprising findings of a multipotent immunomodulator capable of both suppression and enhancement of immune response, depending on the chemical context of the molecule.

The immunomodulator per se is oleylcysteineamide (OCA). On its own or when bound to a carrier (e.g., poly(lactic glycolic) acid (PLGA) nanoparticles) and without any active drug or moiety, it induces and modulates an array of immune responses involving various cytokines. Importantly, the immunomodulatory effects of OCA alone or when bound to a carrier are essentially different in terms of capability to enhance or suppress specific cytokines, thus providing a surprisingly simple and straightforward tool for differential modulation of host immune responses in the context of various clinical conditions.

More specifically, EXAMPLES 1-5 presently demonstrate that the binary system of OCA-carrier (e.g., PLGA nanoparticles, PLGA-NPs) free of any drug agent, induced a stronger immunological response, as per elevated serum Interferon-γ (INF-γ) levels in mice treated with PLGA-NPs compared to CD40L-NPs: CD40L being a known stimulator of INF-γ (FIG. 1).

The OCA-carrier system further induced proliferation of specific populations of immune cells, as per complete blood counts (CBC) showing elevated populations of lymphocytes and monocytes and a reduced population of neutrophils in mice treated with OCA-PLGA-NPs compared to PLGA-NPs treated or untreated mice (FIG. 4). In other words, the immune-activating effect of the OCA-carrier as manifested in vivo in elevated production of the INF-γ cytokine was further accompanied by elevated production of specific populations of immune cells and suppression of other population(s).

In contrast, OCA alone, as a free molecule, inhibited the production of several proinflammatory cytokines in LPS-induced macrophage model in vitro, as per reduced production of TNF-α and IL-6 in the OCA treated cells compared to untreated controls (FIGS. 2-3). The effect was specific and dose dependent and in certain OCA concentrations was comparable to Dexamethasone (DEX).

Further, the capability of OCA to suppress cytokines production was further reproduced in an ex-vivo system of human keratinocytes, where topical application of OCA gel decreased the production of IL-1β and IL-6 to the levels that were comparable and even less than in DEX treated cells as opposed to untreated controls (FIGS. 6-7). The effect was specific and dose-dependent, as per correlation between increasing doses of OCA and decreasing levels of IL-6 and Keratinocyte Chemoattractant (KC)—two typical inflammatory markers in LPS induced keratitis model (FIGS. 5A-5B).

Successful application of OCA to an ex-vivo model of human keratinocytes is particularly attractive, since it implies that OCA can be an effective immunomodulator for topical or ocular applications. Owing to its intrinsic amphiphilic nature, OCA is a likely candidate to penetrate hydrophilic and lipophilic barriers such as skin and cornea.

More generally, it has been presently demonstrated that the functionality of OCA as an immunomodulator was strictly dependent on its structural context, and while as OCA-carrier it was acting as an immune enhancer, OCA alone exhibited activities characteristic of an anti-inflammatory agent. Overall, OCA allows access to a new type of immunotherapies permitting fine-tuning of the host immune responses.

Topical applications of OCA were further explored. EXAMPLE 6 demonstrates surprising and new topical application of OCA as a whitening agent. Accessible skin lightening is an important unmet need in dermatology. A variety of over-the-counter agents are currently available, including Kojic acid, licorice extract and vitamin C. Many of these agents are achieving depigmentation by inhibiting the activity of tyrosinase, being one of the key enzymes in the melanin biosynthesis (melanogenesis) in the epidermal melanocytes. Kojic acid (5-hydroxymethyl-4H-pyran-4-one), a hydrophilic fungal derivative from Aspergillus and Penicillium sp., is relatively efficient whitening agent but is increasingly becoming controversial. Kojic acid inhibits melanin production by binding to copper and inhibiting tyrosinase activity. Its safety is being questioned and raises considerable concerns, owing to which it is banned in some parts of Asia, for example. In other words, there is a pressing need for safer and more efficient whitening agents.

Surprisingly, OCA exhibited the potential to inhibit tyrosinase activity to a similar extent as Kojic acid, as per comparative dose response plots and IC50 estimates on tyrosinase activity in cell-free assay (FIG. 8 and Table 1).

Overall, the results suggest that OCA is a promising and potentially safe candidate to be included in hydrophilic and lipophilic topical formulations either as immunomodulator or as a whitening agent.

### BRIEF DESCRIPTION OF THE DRAWINGS

To better understand the subject matter and to exemplify how it may be carried out in practice, embodiments will now be described by way of non-limiting examples with reference to the following drawings.

FIG. 1 illustrates the immunoenhancing effect of the binary OCA-carrier system. Figure shows serum IFN-γ levels in mice 29 days following IV injection of lymphoma cells and treatment with OCA-PLGA-NPs, as determined by ELISA.

FIG. 2 illustrates the immune-suppressing effect of OCA alone. Figure shows IL-6 level normalized to viability (%) in RAW macrophage 264.7 cells after LPS induction and treatment with various concentrations of OCA (1 and 2.5 μg/ml) and Dexamethasone (DEX, 5 μg/ml).

FIG. 3 illustrates the same effect with the example of TNF-α, using the same system and concentrations of actives.

FIG. 4 illustrates the effect of OCA-PLGA-NPs on the proliferation of specific populations of immune cells. The Figure shows the counts of lymphocytes, monocytes and neutrophils in the peripheral blood of mice treated once a week with PLGA-NPs and PLGA-OCA-NPs (total 3 treatments).

FIGS. 5A-5B illustrate the relationship between OCA dose (0.1%, 0.25%, 0.5% and 1%) and the concentration of IL-6 and Keratinocyte Chemoattractant (KC) in LPS induced keratitis model.

FIG. 6 illustrates the immune-suppressing effect of topical application of OCA on the production of IL-1β in LPS-induced human skin model ex vivo. Figure shows skin preparations treated with OCA gel (1%), DEX (5 μg/ml) and untreated preparations. The results are normalized to LPS-induced untreated skin (*p<0.05).

FIG. 7 illustrates the same effect with the example of IL-6, using the same system and concentrations of actives (*p<0.02, **p<0.01).

FIG. 8 illustrates the effect of OCA as a skin whitening agent. Figure shows the a comparative dose response of tyrosinase activity normalized to non-treated control (%) with various concentrations of OCA and other known whitening agents such as Kojic acid, cysteine and ascorbic acid in a cell-free system.

### DETAILED DESCRIPTION OF EMBODIMENTS

The present invention is essentially centered around oleylcysteineamide (OCA) which was previously recognized as an inert linker moiety for associating various active agents to a surface region of a particle carrier with no measurable biological effects, neither for OCA itself nor for an OCA-associated particle. By this invention, OCA, per se, and OCA-associated particles, per se, have proven effective active pharmaceutical ingredients (APIs) in the absence of other therapeutic agents, and thus could be applicable for treating various types of disorders and clinical or sub-clinical conditions in mammals and humans.

The term ‘active pharmaceutical ingredient (API)’ refers herein to the definition by WHO, i.e., ‘a substance intended to furnish pharmacological activity or to otherwise have direct effect in the diagnosis, cure, mitigation, treatment, or prevention of disease, or to have direct effect in restoring, correcting, or modifying physiological functions in human beings’. As repeatedly stated herein, the API of the invention is OCA, OCA derivative or OCA-associated nano or microparticle. The term API does not encompass any therapeutically or cosmetically known active material.

Oleylcysteineamide (OCA) has the following structure:

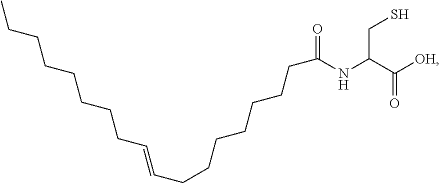 [SMILES: CCCCCCCC/C=C/CCCCCCCC(=O)NC(CS)C(=O)O]

wherein the double bond may be in a cis or trans configuration.

In certain embodiments the invention can use OCT derivatives or analogues.

The term [symbol] derivative [symbol] is used herein under the conventional definition to encompass any compound that is formed from a similar compound or a compound that can be imagined arising from another compound, if one atom is replaced with another atom or group of atoms. The term [symbol] analogue [symbol] (herein also [symbol] homologue [symbol] ) encompasses herein any compound having a structure similar to that of another compound but differing from it in respect to a certain component (also a structural analogue or a chemical analogue).

In certain embodiments the OCT derivatives or analogues can have the general structure (I):

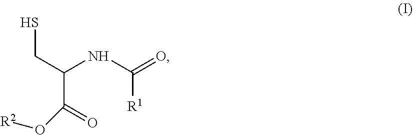

wherein each of R1 and R2, independently of the other, is a lipophilic moiety or H, provided that both R1 and R2 are not H.

In some embodiments each of R1 and R2, independently, can be selected from —H, —C1-C25alkyl, —C2-C25alkenyl, —C2-C25alkynyl, —C6-C10aryl and C3-C10heteroaryl, provided that both R1 and R2 are not H.

In some embodiments, R2 is H.

In some embodiments, R1 is different from H.

In some embodiments, R2 is H and R1 is different from H.

In some embodiments R2 is H and R1 is selected from —C1-C25alkyl, —C2-C25 alkenyl, —C2-C25alkynyl, —C6-C10aryl and C3-C10heteroaryl.

In some embodiments R2 is H and R1 is —C1-C25alkyl or —C2-C25alkenyl.

As used herein, the group —C1-C25alkyl refers to a substituted or unsubstituted, linear or branched aliphatic (alkyl or alkylene) group having between 1 and 25 carbon atoms. Non-limiting examples of such groups include methyl, ethyl, propyl, butyl, pentyl, hexyl, heptyl, octyl, nonyl, decyl, undecyl, dodecyl, tridecyl, tetradyl, pentadecyl, hexadecyl, heptadecyl, octadecyl, nonadecyl, icosanyl and others.

In some embodiments R1 is a group comprising one or more double bonds, thus being of the form —C2-C25alkenyl. The double bond may be positioned along the chain or at the terminus and may be cis or trans. In cases where two or more double bonds are present, they both may be positioned along the chain or one may be at the terminus. They can be both cis or trans, or a mixture of the two configurations.

In some embodiments the moiety R1-C(═O)— is derived from saturated or unsaturated acids and fatty acid, wherein R1 is a fatty chain and —C(═O) designated the carbonyl group of the acid moiety. The saturated or unsaturated or fatty acid may be selected from propionic, butyric, valeric, caproic, enanthic, caprylic, pelargonic, capric, undecylic, lauric, tridecylic, myristic, pentadecylic, palmitic, margaric, stearic, nonadecylic, arachidic, heneicosylic, behenic, tricosylic, lignoceric, pentacosylic, octenoic, decenoic, decadienoic, lauroleic, laurolinoleic, myristovaccenic, myristolinoleic, myristolinolenic, palmitolinolenic, palmitidonic, α-linolenic, stearidonic, dihomo-α-linolenic, eicosatetraenoic, eicosapentaenoic, clupanodonic, docosahexaenoic, 9,12,15,18,21-tetracosapentaenoic, 6,9,12,15,18,21-tetracosahexaenoic, myristoleic, palmitovaccenic, α-eleostearic, β-eleostearic, punicic, 7,10,13-octadecatrienoic, 9,12,15-eicosatrienoic, β-eicosatetraenoic, 8-tetradecenoic, 12-octadecenoic, linoleic, linolelaidic, γ-linolenic, calendic, pinolenic, dihomo-linoleic, dihomo-γ-linolenic, arachidonic, adrenic, osbond, palmitoleic, vaccenic, rumenic, paullinic, 7,10,13-eicosatrienoic, oleic, elaidic, gondoic, erucic, nervonic and 8,11-eicosadienoic acid and others.

In some embodiments the moiety R2O- is derived from an alcohol or a fatty alcohol selected from methyl, ethyl, propyl, butyl, iso-butyl tert-butyl, pentyl, tert-amyl alcohol, hexyl, 3-methyl-3-pentyl, 1-heptyl, capryl, pelargonyl, decyl, capryl, undecyl, lauryl, tridecyl, myristyl, pentadecyl, cetyl, palmitoleyl, heptadecyl, stearyl, oleyl, nonadecyl, arachidyl, heneicosyl, behenyl, erucyl, and lignoceryl alcohol and others.

In a compound of the structure (I), wherein R2 is H and R1 is oleyl, the compound is OCA.

In some embodiments, R2 is H and R1 is any one of propionic, butyric, valeric, caproic, enanthic, caprylic, pelargonic, capric, undecylic, lauric, tridecylic, myristic, pentadecylic, palmitic, margaric, stearic, nonadecylic, arachidic, heneicosylic, behenic, tricosylic, lignoceric, pentacosylic, octenoic, decenoic, decadienoic, lauroleic, laurolinoleic, myristovaccenic, myristolinoleic, myristolinolenic, palmitolinolenic, palmitidonic, α-linolenic, stearidonic, dihomo-α-linolenic, eicosatetraenoic, eicosapentaenoic, clupanodonic, docosahexaenoic, 9,12,15,18,21-tetracosapentaenoic, 6,9,12,15,18,21-tetracosahexaenoic, myristoleic, palmitovaccenic, α-eleostearic, β-eleostearic, punicic, 7,10,13-octadecatrienoic, 9,12,15-eicosatrienoic, β-eicosatetraenoic, 8-tetradecenoic, 12-octadecenoic, linoleic, linolelaidic, γ-linolenic, calendic, pinolenic, dihomo-linoleic, dihomo-γ-linolenic, arachidonic, adrenic, osbond, palmitoleic, vaccenic, rumenic, paullinic, 7,10,13-eicosatrienoic, oleic, elaidic, gondoic, erucic, nervonic and 8,11-eicosadienoic acid, wherein each of the aforementioned groups constitutes a separate embodiment of the invention.

In some embodiments the OCA or the derivative or the analog can be associated with at least one carrier.

In further embodiments the at least one carrier can be a nanocarrier or a microcarrier.

More specifically, the API of the invention consist of OCA, OCA derivative or analogue thereof as defined, or OCA associated to at least one (nano or micro) carrier as defined. Excluded are OCA- or OCA derivative/analogue-carrier compounds wherein either the carrier or the OCA or the derivative or the analogue are associated to another active agent. Also excluded are compounds wherein the carrier comprises or contains or encapsulates another active agent.

The nanocarrier or microcarrier is typically a nanoparticle or a microparticle. Particles composed of materials approved for human or animal use are particularly applicable to the invention, such as materials listed as Generally Recognized as Safe (GRAS) under Sections 201(s) and 409 of the Federal Food, Drug, and Cosmetic Act, and are approved for use in microparticulate systems.

In some embodiments the nanoparticles or microparticles is composed of a polymeric material.

In some embodiments, the polymeric material is selected from poly(lactic acid) (PLA), poly(lacto-co-glycolide) (PLG), poly(lactic glycolic) acid (PLGA), poly(lactide), polyglycolic acid (PGA), poly(caprolactone), poly(hydroxybutyrate) and/or copolymers thereof.

In some embodiments, the polymeric material can be selected from PLA, PGA and PLGA.

In some embodiments, the polymeric material can be PLGA.

The PLGA polymer is a copolymer of polylactic acid (PLA) and polyglycolic acid (PGA), the copolymer being, in some embodiments, selected amongst block copolymer, random copolymer and grafted copolymer.

In some embodiments, the copolymer is a random copolymer.

In some embodiments the PLGA can have an average molecular weight of between 2,000 and 100,000 Da. In other embodiments the PLGA can have an average molecular weight of between 2,000 and 7,000 Da. In other embodiments the PLGA can have an average molecular weight of between 2,000 and 5,000 Da. In still further embodiments the PLGA can have an average molecular weight of between 4,000 and 20,000 Da, or between 4,000 and 10,000 Da, or between 4,000 and 5,000 Da. In still other embodiments the PLGA can have an average molecular weight of about 2,000, about 4,500, about 5,000, about 7,000, about 10,000, about 50,000 or about 100,000 Da.

In some embodiments, the nanoparticles particles can have an average diameter that is between about 10 nm and 1000 nm, and specifically between about 10-100 nm, 100-200 nm, 200-300 nm, 300-400 nm, 400-500 nm, 500-600 nm, 600-700 nm, 700-800 nm, 800-900 nm and 900-1000 nm.

In some embodiments the microparticles can have an average diameter that is between about 10 μm and 1000 μm, and specifically between about 10-100 μm, 100-200 μm, 200-300 μm, 300-400 μm, 400-500 μm, 500-600 μm, 600-700 μm, 700-800 μm, 800-900 μm and 900-1000 μm.

According to the invention, the OCA or the derivatives or the analogues as above can be used as APIs in treating, alleviating or preventing various types of disorders or clinical or sub-clinical conditions in mammals and humans.

In numerous embodiments said disorders or clinical or sub-clinical conditions that comprise inflammation.

In other embodiments said disorders or clinical or sub-clinical conditions or the inflammation can further comprise a microbial infection. The term ‘microbial’ encompasses herein bacterial, vital, fungal and other parasitic infections.

In some embodiments said “treating alleviating or preventing disorders or clinical or sub-clinical conditions” can comprise exerting an anti-inflammatory effect on the disorders or the clinical or sub-clinical conditions.

In some embodiments said exerting an anti-inflammatory effect on the disorders or the clinical or sub-clinical conditions is in the absence of other therapeutic agents, apart from the OCA or a derivative or an analogue and their surface-associated carriers as above.

In other words, according to the invention the API is at least one of the following: (1) OCA; (2) an OCA derivative or analogue as defined herein; or (3) a carrier (e.g., nanoparticle/microparticle) surface-associated with OCA.

In some embodiments the OCA or a derivative or an analogue and their carriers can modulate an immune response. The term [symbol] modulate [symbol] encompasses here a reduction or a suppression of an immune response, or alternatively, an increase or an enhancement of an immune response. The clinical conditions and disorders where the invention can be applicable are detailed further below.

In is another objective of the invention to provide pharmaceutical compositions comprising a therapeutically effective amount of OCA or a derivative or an analogue thereof as an API. In some embodiments the OCA or a derivative or an analogue in such compositions is associated with at least one carrier.

In some embodiments said at least one carrier is a nanocarrier or a microcarrier.

In some embodiments the nanocarrier or the microcarrier is composed of a polymeric material.

In some embodiments the polymeric material is poly(lactic glycolic) acid (PLGA).

In numerous embodiments the OCA or a derivative or an analogue and their carriers can modulate an immune response.

In numerous embodiments the pharmaceutical compositions of the invention can be adapted or formulated to be suitable for various administration modes: oral, enteral, buccal, nasal, topical, transepithelial, rectal, vaginal, aerosol, transmucosal, epidermal, transdermal, dermal, ophthalmic, pulmonary, subcutaneous, intradermal and/or parenteral administration.

In some embodiments the pharmaceutical compositions of the invention can comprise at least one additional therapeutic agent in combination or admixed with the OCA or OCA derivative or analogue. The at least one additional therapeutic agent can be any therapeutic or cosmetic active agent. In such combination of an API according to the invention and at least one additional active agent, the OCA, derivative or homologue thereof or the carrier associated equivalent are not used as carriers but rather as active agents. Thus, such combinations are always mixtures wherein no association is present between an API of the invention and the additional active agent. It should be emphasized that OCA-associated active agents, wherein the active agent is covalently associated to OCA or associated with or contained in a carrier that is associated with OCA are excluded from the present invention.

In numerous embodiments the at least one additional therapeutic agent can be selected from vitamins, proteins, anti-oxidants, peptides, polypeptides, lipids, carbohydrates, hormones, antibodies, monoclonal antibodies, vaccines and other prophylactic agents, diagnostic agents, contrasting agents, nucleic acids, nutraceutical agents, small molecules (molecular weight of less than 1,000 Da or less than 500 Da), electrolytes, drugs, immunological agents and any combination thereof.

The pharmaceutical compositions of the invention can further comprise a pharmaceutically acceptable carrier.

By the same approach, the invention provides cosmetic compositions, cosmeceutical or dermo-cosmetic compositions. This type of compositions should usually include other ranges of effective doses of actives, i.e., OCA or an OCA derivative or analogue.

The term [symbol] effective amount [symbol] or [symbol] effective dose [symbol] broadly relates to an amount of the API of the invention needed to provide a desired level physiological or desirable effect, or improvement of cometic or dermatological condition.

It is another objective of the invention to provide a series of methods for treating, alleviating or preventing disorders or clinical or sub-clinical conditions in mammal and humans, with the main step of administering to the mammal or human a therapeutically effective amount OCA or a derivative or an analogue thereof as API.

The term [symbol] therapeutically effective amount [symbol] (also pharmacologically, pharmaceutically, or physiologically effective amount) broadly relates to an amount of API needed to provide a desired level physiological or clinically measurable response. Analogous terms are [symbol] therapeutic dose [symbol] or [symbol] therapeutically effective dose [symbol] relate to doses of API in a pharmaceutical composition or a dosage form, which can produce an improvement/reduction of at least one symptom of a disorder, a disease or a condition.

In numerous embodiments the methods of the invention are applicable to disorders or clinical or sub-clinical conditions comprising an inflammation.

In numerous embodiments the methods of the invention are applicable to disorders or clinical or sub-clinical conditions and inflammations associated with, mediated by or comprising a microbial infection.

In some embodiments the methods of the invention involve administering OCA or a derivative or an analogue that is associated with at least one carrier.

In some embodiments said at least one carrier is a nanocarrier or a microcarrier.

In some embodiments the nanocarrier or the microcarrier is composed of a polymeric material.

In some embodiments the polymeric material is poly(lactic glycolic) acid (PLGA).

In numerous embodiments the OCA or the derivative or the analogue and their carriers can modulate an immune response.

A special attention should be given to disorders and conditions wherein the compositions and methods of the invention are applicable. One group of such conditions is various inflammatory, infectious and autoimmune disorders. Non-limiting examples of inflammatory diseases that are relevant to the invention (also referred to herein as inflammation or inflammatory condition) include, but not limited to, chronic inflammatory disease and acute inflammatory disease.

Examples for inflammatory diseases include, but not limited to inflammatory diseases associated with hypersensitivity, autoimmune diseases, infectious diseases, graft rejection diseases, allergic diseases and cancerous diseases.

Inflammatory Diseases Associated with Hypersensitivity:

Examples of hypersensitivity include, but are not limited to, Type I, Type II, Type III, and Type IV hypersensitivity, and further immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), Psoriatic Arthritis (PA), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost.2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris) 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus. Note that several same diseases are can be classified to different classes of hypersensitivity, because the heterogeneity of these diseases.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases:

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost.2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris) 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol (1999) 18(1-2):83; Oshima M. et al., Eur J Immunol (1990) 20(12):2563), neuropathies, motor neuropathies (Kornberg A J. J Clin Neurosci. (2000) 7(3):191); Guillain-Barre syndrome and autoimmune neuropathies (Kusunoki S. Am J Med Sci. (2000) 319(4):234), myasthenia, Lambert-Eaton myasthenic syndrome (Takamori M. Am J Med Sci. (2000) 319(4):204); paraneoplastic neurological diseases, cerebellar atrophy, paraneoplastic cerebellar atrophy and stiff-man syndrome (Hiemstra H S. et al., Proc Natl Acad Sci units S A (2001) 98(7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Further, in numerous embodiments the compositions and methods of the invention can be applicable for treating, alleviating and preventing systemic and local conditions and disorders.

In some embodiments they can be applicable to treatment of prevention of chronic granulomatous disease, osteoporosis, Friedreich's ataxia, moderate to severe atopic dermatitis, pulmonary fibrosis or scleroderma.

In some embodiments they can be applicable to treatment of prevention of skin diseases or skin conditions.

In some embodiments they can be applicable to treatment of prevention of hepatitis and tuberculosis.

In some embodiments they can be applicable to treatment of prevention of various types of cancer.

In some embodiments they can be applicable as an adjuvant for cancer immunotherapy.

In some embodiments they can be applicable as an adjuvant for chemotherapies.

In some embodiments they can be applicable as an adjuvant for vaccine therapies.

In some embodiments they can be applicable for invasive fungal infections especially in immunosuppressed patients

In other words, in numerous embodiments the compositions and methods of the invention are applicable for treatment and prevention of disorders or clinical or sub-clinical conditions requiring modulation of an immune response.

In certain embodiments said modulating an immune response comprises inducing or enhancing the immune response. Examples of such conditions are cancer, infectious diseases, inflammatory diseases, and autoimmune diseases, wherein immunostimulatory therapy is needed to detect and eliminate non-self-antigens, and to establish memory effects for these diseases. In other embodiments said modulating an immune response in the mammal comprises reducing or suppressing an inflammation. Examples of such conditions are overactive immune response in diseases like atherosclerosis, rheumatoid arthritis (RA), diabetes, obesity, and transplantation, immunosuppressive therapy is needed to downregulate immune reaction and generate certain immune tolerance.

The mammalian or human immune environment can be regulated by a variety of cytokines to properly control and prevent immune-related disorders or conditions. In numerous embodiments the compositions and methods of the invention can modulate an immune response by modulating the production and/or secretion of at least one cytokine or a cytokine modulator.

In further embodiments said of at least one cytokine or a cytokine modulator is selected from Interferon γ (INF-γ), Tumor Necrosis Factor α (TNF-α), Interleukin 6 (IL-6) and Interleukin 1β (IL-1β).

In some embodiments the composition and methods of the invention use a binary system OCA-carrier such as OCA-PLGA-NPs (nanoparticles) for increasing or enhancing the production and/or secretion of at least one cytokine which is INF-γ.

In some embodiments the composition and methods of the invention use OCA alone for reducing of suppressing the production and/or secretion of at least one cytokine which can be TNF-α, IL-6 or IL-1β.

In numerous embodiments the compositions and methods of the invention can be applied via various administration routes, including oral, enteral, buccal, nasal, topical, transepithelial, rectal, vaginal, aerosol, transmucosal, epidermal, transdermal, dermal, ophthalmic, pulmonary, subcutaneous, intradermal and/or parenteral administrations.

In numerous embodiments the compositions and methods of the invention can include at least one additional therapeutic agent.

The invention can be further articulated in terms of use of OCA or the derivative or analogue and carriers as above as API in the manufacture of a medicament for treating a disorder or a clinical or sub-clinical condition in a mammal.

It is yet another objective of the invention to provide cosmetic, cosmeceutical or dermo-cosmetic compositions and methods.

In this aspect, the invention provides cosmetic compositions comprising an effective amount of Oleylcysteineamide (OCA) or a derivative or an analogue thereof as an active ingredient.

To protect the OCA, OCA derivative or analog or the OCA-associated carrier, as defined herein, from early degradation or ester dissociation, these APIs may be formed into a lyophilized solid powder formulation that may be contained and stored as such and be ready for reconstitution in a liquid carrier upon demand. The liquid carrier may be water-based carrier, for some applications (particularly those for immediate use, e.g., ophthalmic uses), or which may be an anhydrous carrier (water free), such as a silicone-based carrier, for other applications, particularly those necessitating prolonged storage periods. The solid powder may alternatively be used as such, in a non-liquid or formulated form. In some embodiments, the dry powder further comprises at least one cryoprotectant that may optionally be selected from cyclodextrin, PVA, sucrose, trehalose, glycerin, dextrose, polyvinylpyrrolidone, mannitol, xylitol and others. Lyophilization may or may not be carried out in the presence of the at least one cryoprotectant.

Thus, a ready-for-reconstitution powder comprising an API of the invention, as disclosed, is also contemplated herein. The powder may be reconstituted in a liquid carrier, as above, to form a nanoemulsion which may be stable for several weeks or over a period of time defining a treatment regimen. Such products are typically for use as eye or ear products.

A reconstituted formulation comprising an API according to the invention is further provided, which also comprises at least one liquid carrier. As noted, the liquid carrier may be water-based carrier.

The formulation may be for immediate use or for use within a period of between 4 and 28 days, or within a period of time to be prescribed by a medical practitioner. In some embodiments, the formulation is for prolonged use or storage.

Amongst the many types of formulations that can be made using an API of the invention, in some embodiments, such formulations are suitable as ophthalmic formulations configured for injection or as eye drops, or ear formulations, e.g., configured as eardrops.

In another aspect, APIs of the invention or compositions comprising same are suitable for skin whitening.

In certain embodiments such compositions can be used to provide a cosmetic antioxidative effect to the skin. Such compositions are applied locally onto the skin, in other words, they should be adapted for topical or dermal administering. These and also therapeutic compositions of the invention can be administered in a biocompatible aqueous or lipid solution. This solution can be comprised of, but not limited to, saline, water or a pharmaceutically acceptable organic medium.

The cosmetic aspect can be further articulated as methods for skin whitening and/or cosmetic antioxidative effect to the skin in a subject, comprising topical and/or dermal administering to the subject the composition comprising an effective amount of OCA or the derivative or the analog as an active ingredient.

### EXAMPLES

Any method and material similar or equivalent to those described herein can be used in the practice or testing of the present invention. Some embodiments of the invention will be now described by way of examples with reference to respective figures.

### Example 1: OCA-PLGA NPs Conjugate Enhances the Production of INF-γ in Vivo

Methods

Serum interferon-γ (IFN-γ) levels were determined using a commercial sandwich ELISA kit (PeproTech, Rocky Hill, NJ, USA) according to the manufacturer's instructions. Outcomes were quantified by optical density at 450 nm using plate reader (Tecan, Lifesciences). Blood was drawn from NOD/SCID mice at day 29 with the indicated treatment and 5 ul of serum were assayed for IFN-γ levels.

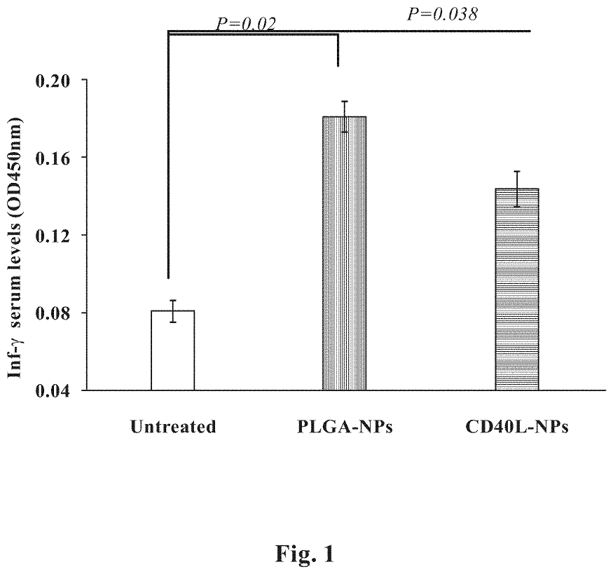

The results showed that a binary system OCA-carrier such as PLGA NPs, and in absence of any drug agent, was capable of inducing a strong immunological response in vivo revealed by elevated serum levels of INF-γ (FIG. 1). The effect was absent when OCA was conjugated with a hydrophilic moiety such as the amino acid cysteine (data not shown).

### Example 2: OCA Alone Suppresses the Production of IL-6 and TNF-α In Vitro

### Preparation of OCA Nanoemulsions

OCA nanoemulsions were prepared at various concentrations via the well-established solvent displacement method (Fessi et al., Int. J. Pharm., 1989, 55:R1-R4). In brief, OCA (10/25/50/100 mg), castor oil (50 mg) and the surfactant Tween 80 (35 mg) were dissolved in acetone (10 mL). The organic phase was then poured into the aqueous phase containing Kolliphor® RH40 (50 mg). The volume ratio between the organic and aqueous phases was 1:2 v/v. The colloidal dispersions were stirred at 900 rpm for 15 min and concentrated by reduced pressure evaporation to 10 mL. 2.5% glycerin w/v were added to the final formulations to obtain isotonic nanoemulsions that were further filtered through 0.22 μm PVDF filter before topical application.

### Size and PDI Characterization

The mean diameter and size distribution of the NEs were measured by Malvern's Zetasizer instrument (Nano series, Nanos-ZS) at 25° C. The formulation (10 μL) was diluted in water (990 μL) and measured in triplicate. The size and PDI remained similar for the 4 tested concentrations respectively 110±5 nm and 0.08±0.01.

### Cell Maintenance and Culture

RAW 264.7 murine macrophage cell line, purchased from American Type Culture Collection (ATCC, USA), was cultured in RPMI 1640 medium (biological industries, Israel) supplemented with 10% (v/v) of heat-inactivated fetal bovine serum and 1% antibiotics (100 U/mL penicillin and 100 μg/mL streptomycin) and incubated at 37° C. in a humidified incubator with 5% CO2. Cell that reached 80% confluency was subcultured and/or used for further experiments. The process of cell detachment involves trypsinization using trypsin enzyme (Biological Industries, Israel).

### Preparation of Assay Plates

The procedure involved the seeding of RAW264.7 cells into 96-well plates with the density of 10000 cells/well. On the next day, when the cells reach confluence, the cells were treated with different concentrations of OCA (1 and 2.5 μg/mL) and dexamethasone (5 μg/mL). All treated and an untreated wells were supplemented with 0.1 μg/mL of LPS (Pseudomonas aeruginosa, Sigma) to induce inflammation. Untreated wells without LPS remained as the control group. Following incubation for 24 h, the culture medium was collected and assayed for measurement of proinflammatory cytokine production, IL6 and TNF-α by ELISA.

### Measurement of Proinflammatory Cytokines Production, IL6 and TNF-α

The proinflammatory cytokines were quantified using the supernatant collected from the treated cells using ELISA (R&D Systems), and the protocol was based on the manual provided in the kit purchased. The measurement of the level of cytokines involved the transferring of the collected cell culture supernatant into four separate 96-well plates coated with the capture antibody against the respective cytokine provided in the kit. The plates were read by using a microplate spectrophotometer at the absorbance of 450 nm.

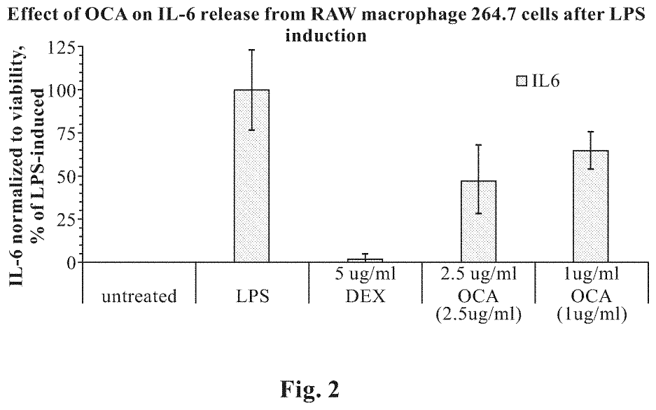

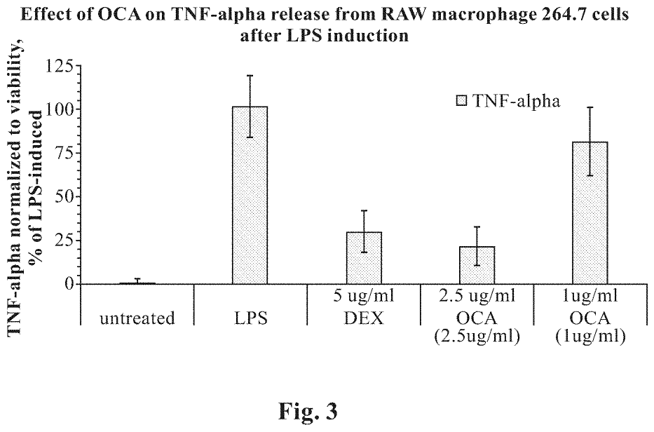

The results showed that OCA alone, as a free molecule, inhibited the production of TNF-α and IL-6 in vitro in LPS-induced macrophage treated with OCA as opposed to untreated controls. The effect was specific and dose dependent, and in certain OCA concentrations was comparable to Dexamethasone (DEX) (FIGS. 2-3).

### Example 3: OCA-PLGA NPs Induce the Proliferation of Specific Populations of Immune Cells In Vivo

Methods

Mice were treated once a week with PLGA-NPs and PLGA-OCA-NPs (total 3 treatments), a day after the third treatment mice peripheral venous blood was obtained from the mouse facial vein using standard techniques and analyzed using the auto hematology analyzer BC-2800 (Mindray) or an automatic Abacus Junior Vet (Diatron), following manufacturer's instructions

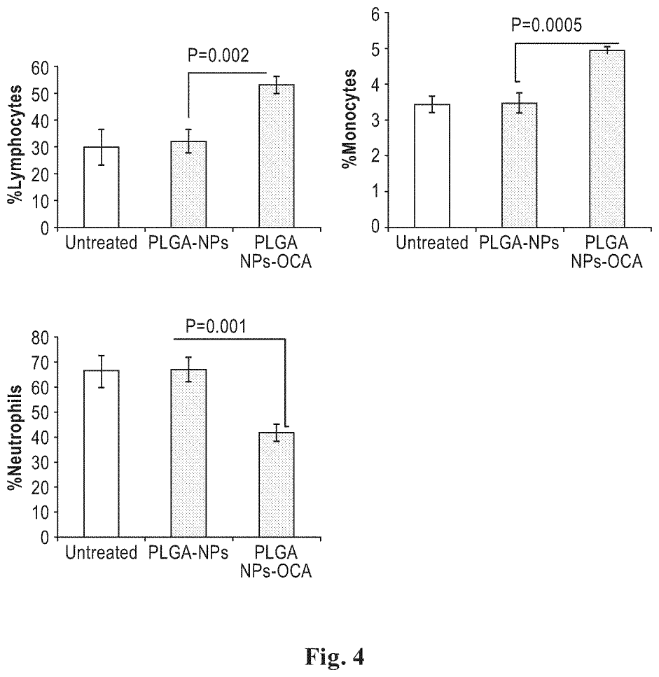

The results showed that the OCA-PLGA-NPs conjugate was capable of inducing the proliferation of specific populations of immune cells, and specifically lymphocytes and monocytes as opposed to neutrophils which was specific to the OCA-PLGA-NPs treated group, but not PLGA-NPs treated or untreated controls (FIG. 4).

### Example 4: OCA Nanoemulsions Suppress Inflammatory Markers in a Keratitis Model In Vivo

### LPS Induced Keratitis Mice Model—Protocol

Mice were maintained in pathogen-free conditions and had free access to food and drinking water. Animals were randomly assigned to the five different study groups. Each time, ten mice were randomly divided into three groups (n=3/4) to evaluate the efficacy of the different treatments in decreasing the inflammation induced by LPS. The results of four combined experiments are presented. All the treatments applied to the mice eyes were adjusted for isotonicity by 2.5% w/v glycerin. 8 weeks female C57BL/6 WT mice were used Intrastromal injection of 0.5 m Pseudomonas aeruginosa LPS in 0.5 μl of PBS performed on all the animals. Mice were treated topically with 2 μL of the various formulations immediately after LPS injections and 1 h after. 24 h after intrastromal injections, mice euthanized, corneas isolated for cytokines analysis.

Treatments groups included:
LPS injected group treated with 2.5% glycerin in sterile water (isotonic vehicle).
0.1% OCA nanoemulsion (NE) with 2.5% glycerin.
0.25% OCA nanoemulsion (NE) with 2.5% glycerin.
0.5% OCA nanoemulsion (NE) with 2.5% glycerin.
1% OCA NE with 2.5% glycerin.

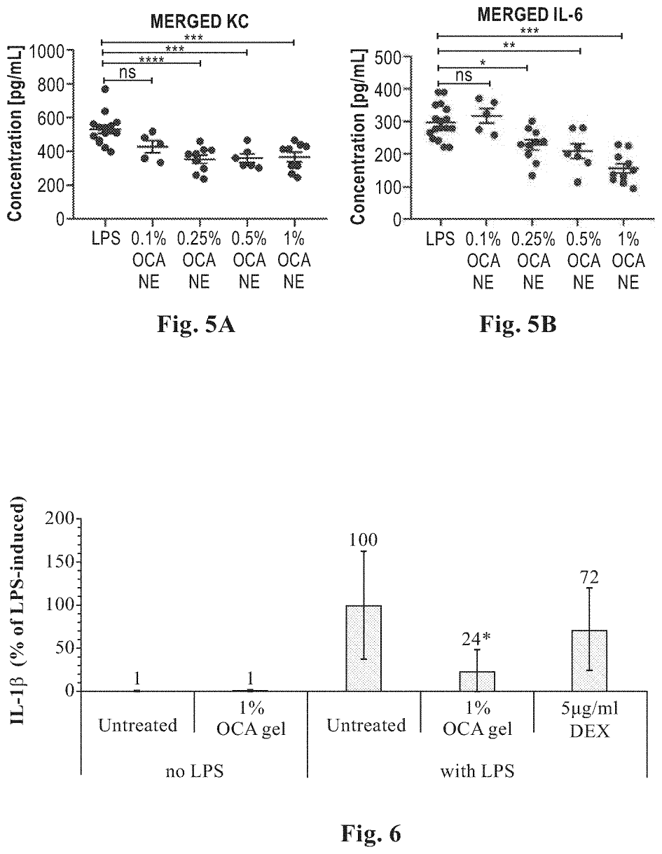

The results showed that increasing concentrations of OCA NE (0.1% to 1%) were correlated to decreasing concentrations of IL-6 and Keratinocyte Chemoattractant (KC)—two typical inflammatory markers (FIGS. 5A-5B).

### Example 5: Topical Application of OCA Suppresses IL-6 and IL-1β in a Keratitis Model Ex-Vivo

Preparation of OCA 1% (w/w) in Pemulen Base

A carbopol base gel formulation (Pemulen) was prepared to test the effect of OCA on the cytokines levels after LPS challenge on skin (ex-vivo)/0.25% w/v pemulen was used for preparing the base gel. Briefly, 50 mg of Pemulen was dispersed in 20 ml. DDW and mixed by an overhead stirrer, further 350 of 1M NaOH were added for obtaining the gel. Fresh OCA 1% gel, was prepared by addition of 10 mg of OCA dissolved in 25 μl ethanol to one gram of pemulen gel base, stirred to get homogenous gel.

### Effect of Topically Applied OCA Gel on LPS-Induced Inflammatory Skin Model Ex Vivo

Fresh normal human skin, obtained from elective plastic surgery (abdominoplasty) was freed from underlying fat, cut into 0.5*0.5 cm pieces and sterilized by soaking for 1 minute in 70% ethanol. Skin explants were cultured in 24-well plates with dermis immersed in culture medium (0.35 mL) supplemented with LPS to induce inflammation [5 μg/mL (E. coli, Santa Cruz)].

Epidermis, exposed to air, was smeared with a thin layer of the test formulation, corresponding to the applied weight of 1-2 mg/cm2. In 1% gel preparation, this amount contained 10-20 μg of active/cm2. In the experiments, the skin piece surface was 0.25 cm2, meaning 2.5-5 μg of active was topically applied to the sample. The positive control, 5 μg/mL of dexamethasone dissolved in 0.35 mL medium, contained 1.75 μg of the drug per sample. Untreated pieces without LPS remained as the negative control. The results were from one donor, performed with 5-6 replicates. After 24-hour treatment, cell culture medium was collected and assayed for the levels of proinflammatory cytokines IL-1β and IL-6 by ELISA specific kits (BioLegend).

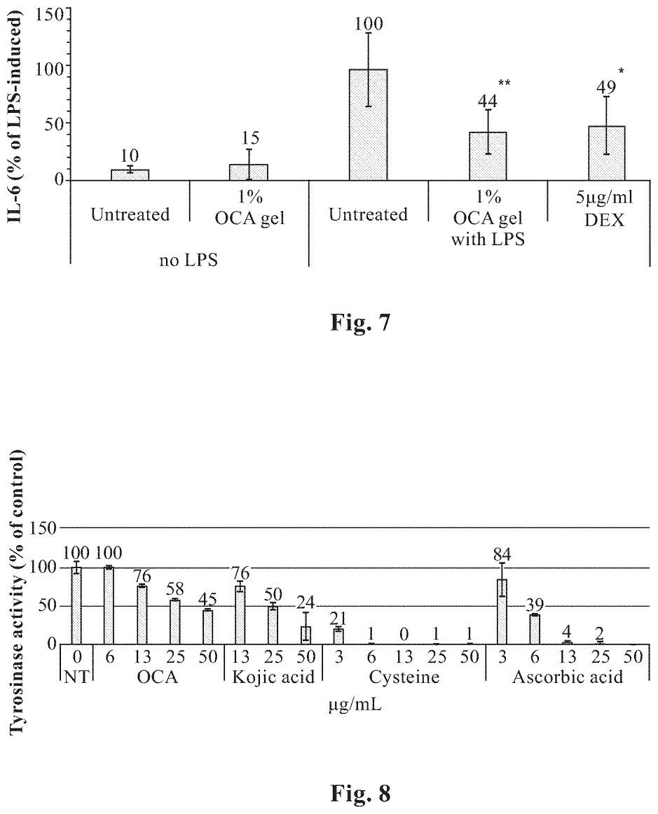

The results showed that under certain conditions and doses, OCA can provide immunomodulatory effects that are comparable to DEX (FIGS. 6-7). Overall, the results suggested that due to its amphiphilic nature and its potential to penetrate cell membranes, OCA could provide an effective agent to suppress inflammation in different tissues.

### Example 6: OCA as Skin-Whitening Agent Inhibiting Tyrosinase In Vitro

Methods

Tyrosinase activity was tested in a cell-free assay as a function of DOPA oxidase activity. The assay is a modification of the previously described assay (Oh et al, Ann Dermatol., 2014, 26(6):681-7). To 10 μL sample (in a 96-well plate) was added 95 μl phosphate buffer saline containing 10 μg/mL mushroom tyrosinase (Worthington, Lakewood, NJ, USA, 505 U/mg). After incubations for 10 min at RT, enzymatic reaction was initiated by adding 95 μl of phosphate buffer saline containing 1 mM L-DOPA. Absorbance values were measured every 5 minutes for 40 min at 475 nm using an ELISA reader at an incubation temperature of 37° C. The quantity of dopachrome formed in the reaction mixture was determined against a blank (solution without enzyme) at 475 nm in the ELISA reader and expressed as a slope of the enzyme activity, measured in the linear part of the curve, normalized to the untreated control wells.

To evaluate the efficacy of OCA as a depigmenting agent, the effect of OCA was compared to known whitening agents such as cysteine, Kojic acid (5-hydroxymethyl-4H-pyran-4-one) and ascorbic acid using comparative dose-dependent response on tyrosinase activity (FIG. 8) and IC50 estimates on tyrosinase activity (Table 1)

TABLE 1
Calculated IC₅₀ (μg/mL) on tyrosinase activity

| OCA | Kojic acid | Cysteine | Ascorbic acid |
|---|---|---|---|
| 33.8 | 25.12 | 2.41 | 5.39 |

The results showed that OCA had a similar whitening effect as Kojic acid in terms of inhibition of tyrosinase. Overall, OCA can provide a potentially safe new whitening agent to be included in topical skin formulations.

1.-52. (canceled)

53. Oleylcysteineamide (OCA) for use as an active pharmaceutical ingredient (API) in treating, alleviating or preventing a disorder or a clinical or sub-clinical condition in a mammal, wherein the OCA is not associated to a therapeutically or a cosmetically active agent.

54. The OCA of claim 53, wherein said disorder or clinical or sub-clinical condition comprises an inflammation.

55. The OCA claim 53, wherein said disorder or clinical or sub-clinical condition or the inflammation further comprises a microbial infection.

56. The OCA of claim 53, wherein said treating alleviating or preventing a disorder or a clinical or sub-clinical condition comprises exerting an anti-inflammatory effect on the disorder or the clinical or sub-clinical condition.

57. The OCA claim 56, wherein said exerting an anti-inflammatory effect on the disorder or the clinical or sub-clinical condition is in the absence of other therapeutic agents.

58. The OCA claim 53, wherein the OCA modulates an immune response.

59. The OCA of claim 53, wherein the OCA is associated with at least one carrier.

60. The OCA of claim 59, wherein said at least one carrier is a nanocarrier or a microcarrier.

61. The OCA of claim 60, wherein the nanocarrier or the microcarrier is composed of a polymeric material.

62. The OCA of claim 61, wherein the polymeric material is poly(lactic glycolic) acid (PLGA).

63. A method of treating, alleviating or preventing a disorder or a clinical or sub-clinical condition in a mammal, the method comprising administering to the mammal a therapeutically effective amount of oleylcysteineamide (OCA) as an active pharmaceutical ingredient (API), wherein the OCA is not associated to a therapeutically or a cosmetically active.

64. The method of claim 63, wherein said disorder or clinical or sub-clinical condition comprises an inflammation.

65. The method of claim 63, wherein said wherein said disorder or clinical or sub-clinical condition or the inflammation further comprises a microbial infection.

66. The method of claim 63, wherein said OCA is associated with at least one carrier.

67. The method of claim 66, wherein said at least one carrier is a nanocarrier or a microcarrier.

68. The method of claim 67, wherein the nanocarrier or the microcarrier is composed of a polymeric material.

69. The method of claim 68, wherein the polymeric material is poly(lactic glycolic) acid (PLGA).

70. A pharmaceutical or a cosmetic composition comprising an effective amount of Oleylcysteineamide (OCA) as an active ingredient, wherein the OCA is not associated to a therapeutically or a cosmetically active agent.

71. A method for skin whitening and/or cosmetic antioxidative effect to the skin in a subject, the method comprising topical and/or dermal administering to the subject the composition comprising an effective amount of Oleylcysteineamide (OCA) as an active ingredient, wherein the OCA is not associated to a therapeutically or a cosmetically active agent.

72. An active pharmaceutical ingredient (API) selected from (i) oleylcysteineamide (OCA), (ii) a nanocarrier associated with OCA, and (iii) a microcarrier associated with OCA; wherein the API is not associated to a therapeutically or a cosmetically active agent.

73. A reconstituted formulation comprising an API according to claim 72 and at least one liquid carrier, wherein the formulation being optionally being an ophthalmic formulation configured for injection or as eye drops, or being configured as eardrops.
